(19) United States
(12) Patent Application Publication
(10) Pub. No.: US 2025/0319100 A1
(43) Pub. Date: Oct. 16, 2025

(54) ANTI-TUMOR COMBINED PREPARATION COMPRISING HYDROXYPROGESTERONE CAPROATE AND USE THEREOF

(71) Applicant: SHENZHEN EVERGREEN THERAPEUTICS CO., LTD., Shenzhen, Guangdong (CN)

(72) Inventors: Tao Tom DU, Shenzhen City (CN); Tao HU, Shenzhen City (CN); Yuqian ZHANG, Shenzhen City (CN); Xin DU, Shenzhen City (CN)

(73) Assignee: SHENZHEN EVERGREEN THERAPEUTICS CO., LTD., Shenzhen, Guangdong (CN)

(21) Appl. No.: 18/572,681

(22) PCT Filed: Mar. 11, 2022
(86) PCT No.: PCT/CN2022/080275

§ 371 Date: Dec. 20, 2023

(30) Foreign Application Priority Data
Mar. 11, 2021 (CN) 202110265778.5

(51) Int. Cl. A61K 31/57; A61K 39/00; A61K 39/395; A61P 35/04

(52) U.S. Cl. CPC A61K 31/57; A61K 39/39558; A61P 35/04; A61K 2039/505

(57) ABSTRACT

Provided in the present invention is a pharmaceutical composition comprising hydroxyprogesterone caproate and an additional therapeutic agent for treating tumors, wherein the additional therapeutic agent is preferably a monoclonal antibody against PD-1 or PD-L1. Further provided in the present invention are a method for increasing the number of tumor infiltrating lymphocytes (TIL) as required and a method for treating cancers in a subject in need thereof, which comprises providing the hydroxyprogesterone caproate to the subject.

### CROSS REFERENCE TO RELATED APPLICATION

The present application claims the priority of the Chinese patent application filed with the China National Intellectual Property Administration on Mar. 11, 2021, with application No. 202110265778.5, titled “Anti-Tumor Combination Preparation Containing Hydroxyprogesterone Acetate and Its Applications.” The entire content thereof is incorporated herein by reference in this application.

### TECHNICAL FILED

The present invention relates to a pharmaceutical composition for treating tumors and a method for treating tumors, particularly involving a pharmaceutical composition that enhances the effectiveness of tumor therapeutics by improving the tumor microenvironment and a method for treating tumors through combination therapy.

### BACKGROUND

Malignant tumors (cancer) pose a significant threat to human health. Conventional treatment methods include surgery, radiation therapy, chemotherapy, and targeted therapy. However, their effectiveness against advanced-stage tumors is limited and often accompanied by substantial toxicity and poor tolerability issues. In recent years, tumor immunotherapy has shown remarkable clinical efficacy. It is a treatment modality that activates the body's own immune system to combat tumors and is considered the most promising approach for curing malignant tumors. As such, tumor immunotherapy was honored with the Nobel Prize in Physiology or Medicine in 2018.

The tumor microenvironment is a crucial aspect of cancer biology, contributing to tumor initiation, progression, and responses to treatment. Cells and molecules of the immune system constitute fundamental components of the tumor microenvironment. The composition and characteristics of the tumor microenvironment vary significantly and play a vital role in determining the anti-tumor immune response. For instance, certain immune system cells, including natural killer cells, dendritic cells (DCs), and effector T cells, can drive robust anti-tumor responses. However, tumor cells often induce an immunosuppressive microenvironment, favoring the development of immunosuppressive populations of immune cells, such as myeloid-derived suppressor cells and regulatory T cells.

Immune checkpoint inhibitors, as representatives of tumor immunotherapy, function by blocking immunosuppressive signals in the tumor microenvironment, restoring the functionality of immune cells to exert anti-tumor effects. Among these, the most extensively researched and clinically applied immune checkpoints include programmed cell death protein 1 (PD-1), programmed death-ligand 1 and 2 (PD-L1/L2), cytotoxic T-lymphocyte-associated protein 4 (CTLA-4), lymphocyte activation gene 3 (LAG-3), T-cell immunoglobulin and mucin domain-containing protein 3 (TIM-3), T-cell immunoglobulin and ITIM domain (TIGIT), and V-domain Ig suppressor of T-cell activation (VISTA) (Randolph J. Nolle, Science, 2020).

In the tumor microenvironment, PD-1 and/or PD-L1 are highly expressed in most tumor-infiltrating lymphocytes (TILs) from various tumor types. They inhibit localized immune responses, including lung cancer, liver cancer, ovarian cancer, cervical cancer, skin cancer, colon cancer, glioma, bladder cancer, breast cancer, kidney cancer, esophageal cancer, stomach cancer, oral squamous cell carcinoma, urothelial carcinoma, pancreatic cancer, and head and neck tumors. PD-1 and/or PD-L1 antibodies can block the binding of PD-L1 on tumor cells with the inhibitory receptor PD-1 on the surface of activated T cells, thereby activating tumor-specific T cells and restoring the immune system's ability to eliminate tumor cells. However, due to the substantial heterogeneity and genetic instability of tumors, their complex pathogenesis leads to various endogenous and exogenous immune resistance mechanisms. PD-1 antibodies exhibit a clinical response rate of only about 20%.

The inventors discovered through research that not only PD-1 antibodies but also other antibodies and various tumor therapeutics show significant differences in clinical efficacy compared to in vitro experiments. An important reason for this discrepancy is the tumor's microenvironment, which is conducive to cancer cell survival and proliferation. This results in challenges such as drugs struggling to reach their targets and immune cells finding it difficult to penetrate tumor tissues to eliminate cancer cells.

17α-Hydroxyprogesterone caproate, also known as hydroxyprogesterone caproate, 17α-hydroxyprogesterone acetate, 17-OHPC, OHPC, with the chemical formula C27H40O4 and CAS No. 630-56-8, is clinically used for treating habitual abortion, menstrual disorders, endometriosis, functional uterine bleeding, etc.

### SUMMARY OF THE INVENTION

According to one aspect of the present invention, a pharmaceutical composition is provided, comprising hydroxyprogesterone caproate and an additional therapeutic agent for treating tumors.

Optionally, the additional therapeutic agent for treating tumors is selected from: chemotherapeutic agents, targeted therapeutic agents, hormonal therapeutic agents, cellular therapeutic agents, oncolytic viral agents, or antibodies, particularly immune checkpoint inhibitors.

Optionally, the immune checkpoint inhibitor is selected from inhibitors of PD-L1, PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, SLAM, or combinations thereof. Preferably, the immune checkpoint inhibitor is a monoclonal antibody, particularly targeting PD-1 or PD-L1.

Optionally, the additional therapeutic agent is selected from atezolizumab, avelumab, durvalumab, ceralasertib, cemiplimab, ipilimumab, nivolumab, pembrolizumab, sintilimab, tremelimumab, tislelizumab, toripalimab, tiragolumab, or RC98.

According to another aspect of the invention, an anti-tumor drug is provided, comprising the aforementioned pharmaceutical composition containing hydroxyprogesterone caproate and another therapeutic agent for treating tumors, and optionally one or more pharmaceutically acceptable excipients.

Optionally, the formulation of the anti-tumor drug is selected from injectable, oral, or topical formulations; preferably, an injectable formulation; particularly preferably, an injectable liquid or powder for injection.

Optionally, the content of the additional therapeutic agent is 10%-100% of the effective dose when used alone. For instance, in a daily tablet/dose formulation, the content of hydroxyprogesterone caproate is 100-1000 mg per tablet/dose; for a formulation with m tablets/doses used daily, the content of hydroxyprogesterone caproate is (100-1000 mg)/m per tablet/dose; for a formulation used every n days, the content of hydroxyprogesterone caproate is n×(100-1000 mg) per tablet/dose.

Optionally, the pharmaceutically acceptable excipients include solvents, co-solvents, solubilizers, emulsifiers, colorants, binders, disintegrants, fillers, lubricants, wetting agents, osmotic pressure regulators, stabilizers, flow aids, masking agents, preservatives, suspending agents, coating materials, fragrances, anti-adherents, integrators, permeation enhancers, pH regulators, buffers, plasticizers, surfactants, thickeners, encapsulating agents, moisturizers, absorbents, diluents, flocculating agents, anti-flocculating agents, aid filters, release retardants, high molecular weight skeleton materials, and film-forming materials, at least one of which is used.

According to another aspect of the invention, a method is provided for increasing the number of tumor-infiltrating lymphocytes (TILs) in a subject when needed, comprising providing hydroxyprogesterone caproate to the subject.

Optionally, the TIL population comprises CD8+ T cells, CD4+ T cells, B cells, natural killer (NK) cells, dendritic cells (DCs), M1 macrophages, or combinations thereof, preferably expressing the TH1 phenotype of T cells.

Optionally, the increase in TIL number occurs in the tumor microenvironment (TME) and/or within the tumor tissue.

According to another aspect of the invention, a method is provided for treating cancer in a subject in need, comprising providing hydroxyprogesterone caproate to the subject.

Optionally, the method includes providing an additional therapeutic agent for treating cancer, apart from hydroxyprogesterone caproate.

Optionally, the additional therapeutic agent is selected from chemotherapeutic agents, targeted therapeutic agents, hormonal therapeutic agents, cellular therapeutic agents, oncolytic viral agents, or antibodies.

Optionally, the additional therapeutic agent is selected from immune therapeutic agents.

Optionally, the additional therapeutic agent is selected from immune checkpoint inhibitors.

Optionally, the immune checkpoint inhibitor is selected from inhibitors of PD-L1, PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, SLAM, preferably targeting PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, SLAM, or combinations thereof, with monoclonal antibodies particularly targeting PD-L1 or PD-1.

Optionally, the additional therapeutic agent is selected from atezolizumab, avelumab, durvalumab, ceralasertib, cemiplimab, ipilimumab, nivolumab, pembrolizumab, sintilimab, tremelimumab, tislelizumab, toripalimab, tiragolumab, or RC98.

Optionally, the subject has high PD-L1 or PD-1 expression.

Optionally, it is applied as a first-line or second-line treatment method.

Optionally, hydroxyprogesterone caproate can be administered to the subject before, simultaneously, or after administering the additional therapeutic agent.

Optionally, the subject suffers from primary or metastatic cancer. The cancer includes solid tumors or metastatic cancers, where solid tumors are bladder cancer, bone cancer, brain cancer, breast cancer, colorectal cancer, endometrial cancer, esophageal cancer, eye cancer, head and neck cancer, kidney cancer, lung cancer, melanoma, ovarian cancer, pancreatic cancer, prostate cancer, or gastric cancer. Preferably, breast cancer, lung cancer, renal cell carcinoma (RCC), hepatocellular carcinoma (HCC), melanoma, colorectal cancer, or endometrial cancer, particularly primary or metastatic non-small cell lung cancer (NSCLC), and the metastatic cancer includes metastatic melanoma, metastatic colorectal cancer, or metastatic endometrial cancer.

Optionally, the subject suffers from recurrent cancer or refractory cancer.

Optionally, using hydroxyprogesterone caproate or using hydroxyprogesterone caproate with an additional therapeutic agent for treating cancer reduces tumor cell proliferation in the subject and/or slows down or stabilizes tumor growth.

### BRIEF DESCRIPTION OF THE DRAWINGS

To provide a clearer explanation of specific embodiments of the invention or technical solutions in the prior art, a brief introduction to the drawings required for the description of specific embodiments or prior art will be provided below. It is evident that the illustrations in the description below represent certain embodiments of the present invention. Those skilled in the art can derive other illustrations based on these illustrations without exercising inventive labor.

FIG. 1 depicts the trend of tumor volume growth after administration.

FIG. 2 illustrates the infiltration of CD8 T cells in mouse tumors post-administration (400× magnification).

FIG. 3 presents the analysis of CD8 staining density on tumor sections after administration, represented in integrated optical density (IOD)/area, where ** denotes p<0.01 compared between the OHPC group and the control group.

### DETAILED DESCRIPTION

The provided embodiments are meant to enhance the understanding of the present invention and are not limited to the described best embodiments. They do not restrict the content and scope of the invention. Any product obtained by anyone under the guidance of the present invention or by combining the features of the present invention with other features of the prior art falls within the protection scope of the present invention.

If specific experimental steps or conditions are not indicated in the embodiments, the operations or conditions can be carried out according to the conventional experimental procedures described in the literature within this field. Reagents or instruments not specified by manufacturers are standard products available for purchase in the market.

### Example 1

### 1. Investigational Drugs and Reagents

Antibody Anti-PD-L1 was purchased from Rongchang Biopharmaceuticals (Yantai) Co., Ltd., batch number: RC98-1-A20170426, with a storage solution concentration of 6.87 mg/ml, dissolved in 0.9% sodium chloride solution. Prior to each administration, the Anti-PD-L1 was prepared by diluting it in 0.9% sodium chloride solution to obtain a concentration of 1 mg/ml.

Hydroxyprogesterone Caproate (OHPC) was purchased from Hongyan Pharmaceutical and Chemical Co., Ltd., as a powder. Prior to each administration, OHPC powder was dissolved in DMSO. Once fully dissolved, it was diluted in sesame oil to obtain an OHPC concentration of 4 mg/ml for the investigational drug solution.

### 2. Animal Grouping, Modeling, and Drug Administration

### (1) Cell Culture

Murine breast cancer 4T1 cells were purchased from Beijing Biosynthgene Biotech Co., Ltd. Cells were cultured at 37° C. and 5% CO2 in DMEM culture medium containing 10% inactivated fetal bovine serum. The 4T1 cells were genetically modified using conventional methods to overexpress human PD-L1, and these cells were named 4T1-hPD-1.

### (2) Tumor Cell Inoculation, Grouping, and Drug Administration

Female Balb/c mice (6-9 weeks old, weighing 16-22 g), SPF grade, were subcutaneously inoculated with 5×10⁴ cells/0.1 ml concentration of 4T1-hPD-1 cells suspended in PBS at a volume of 0.1 ml on the right side. When the average tumor volume reached approximately 100 mm3, appropriate mice were selected based on tumor volume and weight and were evenly distributed into four experimental groups, each with 8 mice. Drug administration commenced on the same day as outlined in the table below:

TABLE 1
Drug Administration

| | Test Product, Dose | Animal | | Number of |
|---|---|---|---|---|
| Group | (mg/kg)^{a} | Number | Frequency | Administration |
| Control Group(G1) | PBS (Intramuscular) | 8 | Twice per | 6 |
| | | | week | |
| Anti-PD-L1(G2) | Anti-PD-L1 (10 mg/kg, | 8 | Twice per | 6 |
| | Intravenous) | | week | |
| OHPC(G3) | OHPC (20 mg/kg, | 8 | Twice per | 6 |
| | Intramuscular) | | week | |
| Anti-PD- | Anti-PD-L1 (10 mg/kg, | 8 | Twice per | 6 |
| L1 + OHPC(G4) | Intravenous) + OHPC | | week | |
| | (20 mg/kg, | | | |
| | Intramuscular) | | | |

^{a}The dosage volume was calculated at 10 μl/g of the experimental animal's volume.

After the final administration, the body weight and tumor growth status of the experimental animals were observed for an additional 12 days. Throughout the observation period, tumor volume and animal weight were measured weekly, and the values were recorded. Upon completion of the observation period, the experiment was concluded.

### 3. Measurement Parameters

### (1) Tumor Volume

Measurements of tumor volume were conducted twice a week post-grouping using a vernier caliper. Prior to euthanasia, tumor dimensions including the longest and shortest diameters were measured. The formula used to calculate tumor volume: Tumor Volume=0.5×Longest Diameter×Shortest Diameter².

### (2) Body Weight Monitoring

Animals were weighed at various intervals: upon inoculation, pre-dosing (i.e., before the initial administration), twice a week during drug administration, and before euthanasia.

### (3) General Clinical Observations

Daily observations were carried out throughout the adaptation period and the experimental phase. Observations included but were not limited to monitoring tumor nodules for ulceration, assessing the animals' behavioral and dietary patterns.

### 4. Evaluation Criteria for Drugs

### (1) Tumor Growth Inhibition Rate (TGITV)

$${TGl}_{TV} ( \% ) = [ 1 - ( Ti - T 0 ) / ( Vi - V 0 ) ] \times 100 \% ;$$

Ti represents the mean tumor volume of the treatment group on day i of drug administration, TO denotes the mean tumor volume of the treatment group on day 0 of drug administration. Vi stands for the mean tumor volume of the solvent control group on day i of drug administration, and VO indicates the mean tumor volume of the solvent control group on day 0 of drug administration.

### (2) Infiltration of CD8+ T Cells in Tumor Tissue

At the conclusion of the experiment, tumor tissue was embedded in paraffin, sectioned, and stained for CD markers. This was conducted to compare the infiltration of CD8+ T cells within tumors across different groups.

### 5. Data Collection and Statistical Analysis

Original data from observations and measurements were recorded. Analysis and processing were based on this raw data. Results were expressed as Mean±Standard Error of Mean (SEM). Statistical analysis was performed on tumor volume data, where P<0.05 was considered statistically significant. Analysis was conducted considering both statistical and biological significance.

### 6. Experimental Results

### (1) Tumor Volume Inhibition Results

FIG. 1 shows the trend of tumor volume growth post-administration. As depicted in FIG. 1, 4T1 breast cancer demonstrated resistance to immunotherapy, hence the anti-PD-L1 group didn't exhibit any significant tumor growth inhibition. Similarly, the administration of OHPC alone showed no inhibitory effect. However, in the combination group of anti-PD-L1 and OHPC, there was a noticeable suppression of tumor growth in the later stages. By the 17th day, the experiment's conclusion, the inhibition rate reached 30.7%.

Results for tumor volume and tumor volume inhibition rates for each group are listed in Table 2.

TABLE 2
Effect of Test Products on Tumor Volume in
Balb/c Mice Injected with 4T1-hPD-L1 Cells

| | | Tumor Volume (mm³)^{a} | | | |
|---|---|---|---|---|---|
| | | Pre- | | | |
| Group | Test Product | administration | Day 17 | TGl_{TV}(%) | P^{b} |
| G1 | Vehicle(PBS) | 84 ± 27 | 1062 ± 245 | — | — |
| G2 | Anti-PD-L1 | 89 ± 18 | 1116 ± 234 | −4.9 | 0.725 |
| G3 | OHPC | 87 ± 24 | 1203 ± 384 | −14 | 0.398 |
| G4 | Anti-PD- | 88 ± 18 | 766 ± 81 | 30.7 | 0.016 |
| | L1 + OHPC | | | | |

Note:
^{a}Mean ± Standard Error
^{b}Comparison of group weight to Vehicle control weight on the 17th day of dosing was analyzed using t-test.

### (2) Infiltration of CD8+ T Cells in Tumor Tissue

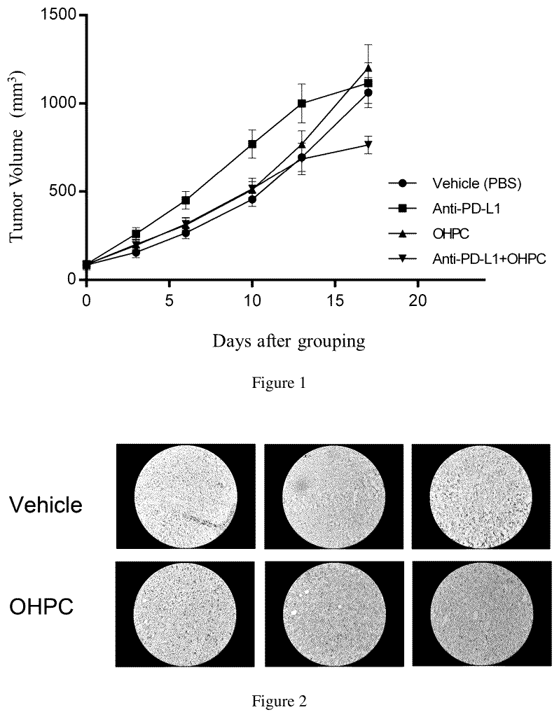

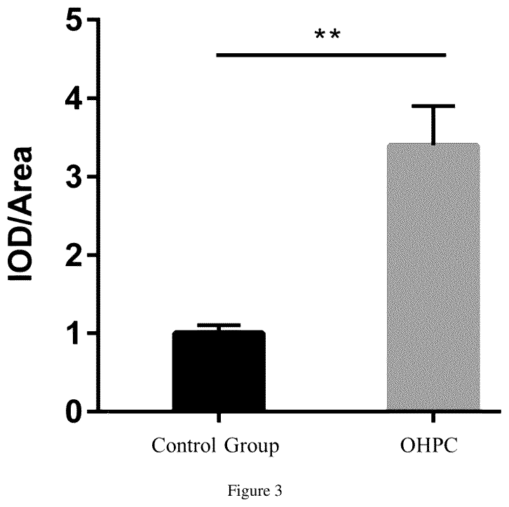

Refer to Supplementary FIGS. 2 and 3 in the manual; Hydroxyprogesterone Caproate (OHPC) notably enhanced the infiltration of CD8+ T cells.

### 7. Result

The hydroxyprogesterone caproate significantly enhances the infiltration of CD8+ T cells and augments the anti-tumor activity of the monoclonal antibody Anti-PD-L1.

Evidently, the embodiments are presented solely for illustrative purposes and do not limit the implementation. Ordinary skilled practitioners in this field may introduce other modifications or variations based on the provided descriptions. It is not necessary nor feasible to exhaustively list all possible embodiments. The evident variations or alterations derived from this remain within the scope of protection provided by this invention.

1. A pharmaceutical composition comprising hydroxyprogesterone acetate and an additional therapeutic agent for treating tumors.

2. The pharmaceutical composition of claim 1, wherein the additional therapeutic agent for treating tumors is selected from: chemotherapeutic agents, targeted therapy agents, hormonal therapy agents, cell therapy agents, oncolytic viral agents, or antibodies; preferably, the additional therapeutic agent is an immune checkpoint inhibitor.

3. (canceled)

4. The pharmaceutical composition of claim 32, wherein the immune checkpoint inhibitor targets PD-L1, PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM, preferably a monoclonal antibody targeting PD-1 or PD-L1.

5. The pharmaceutical composition of claim 4, wherein the additional therapeutic agent is selected from atezolizumab, avelumab, durvalumab, cemiplimab, siltuximab, ipilimumab, nivolumab, pembrolizumab, sintilimab, tislelizumab, tremelimumab, tisotumab vedotin, or RC98.

6. An anti-tumor drug comprising the pharmaceutical compositions of claim 1 and optionally one or more pharmaceutically acceptable excipients.

7. The anti-tumor drug of claim 6, wherein the drug formulation is selected from injectable formulations, oral formulations, or topical formulations; preferably injectable or oral formulations; particularly preferably injectable formulations; more preferably, the injectable formulation is selected from injectable solutions or powder for injection; the oral formulation is selected from tablets, solutions, capsules, powders, pills, granules, syrups, suspensions, or orally disintegrating controlled-release formulations; the topical formulation is selected from ointments, sprays, or patches.

8. (canceled)

9. The anti-tumor drug of claim 6, wherein the content of the additional therapeutic agent is 10%-100% of the effective dose for individual use.

10. The anti-tumor drug of claim 6, wherein in a once-a-day tablet/dose formulation, the content of hydroxyprogesterone acetate per tablet/dose is 100-1000 mg; in a formulation used m times daily, the content of hydroxyprogesterone acetate per tablet/dose is (100-1000 mg)/m; in a formulation used once every n days, the content of hydroxyprogesterone acetate per tablet/dose is n×(100-1000) mg.

11. A method of increasing the number of tumor-infiltrating lymphocytes (TIL) when needed, comprising administering hydroxyprogesterone acetate to a subject.

12. The method of claim 11, wherein the TIL population comprises a group of CD8+ T cells, CD4+ T cells, B cells, natural killer (NK) cells, dendritic cells (DCs), M1 macrophages, or a combination thereof, preferably expressing a TH1 phenotype of T cells; or
wherein the increase in TIL numbers occurs in the tumor microenvironment (TME) and/or within tumor tissues.

13. (canceled)

14. A method for treating cancer in a subject in need thereof, comprising administering hydroxyprogesterone acetate to the subject.

15. The method as claimed in claim 11, further comprising administering an additional therapeutic agent for treating cancer to the subject, apart from hydroxyprogesterone acetate.

16. The method as claimed in claim 15, wherein the additional therapeutic agent is selected from chemotherapeutic agents, targeted therapy agents, hormonal therapy agents, cell therapy agents, oncolytic viral agents, or antibodies; or the additional therapeutic agent is an immune therapy agent; preferably, the additional therapeutic agent is an immune checkpoint inhibitor.

17. (canceled)

18. (canceled)

19. The method as claimed in claim 16, wherein the immune checkpoint inhibitor is selected from inhibitors of PD-L1, PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, SLAM, or a monoclonal antibody targeting PD-1, PD-L2, CTLA-4, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, SLAM, or a combination thereof, particularly preferably a monoclonal antibody targeting PD-L1 or PD-1.

20. The method as claimed in claim 15, wherein the additional therapeutic agent is selected from atezolizumab, avelumab, durvalumab, cemiplimab, siltuximab, ipilimumab, nivolumab, pembrolizumab, sintilimab, tislelizumab, tremelimumab, tisotumab vedotin, or RC98.

21. The method as claimed in claim 15, wherein the subject has higher expression levels of PD-L1 or PD-1.

22. The method as claimed in claim 11, used as a first-line or second-line treatment method.

23. The method as claimed in claim 15, wherein hydroxyprogesterone acetate is administered to the subject before, simultaneously with, or after administering the additional therapeutic agent.

24. The method as claimed in claim 11, wherein the subject has primary or metastatic cancer; preferably, the cancer is a solid tumor or metastatic cancer, wherein the solid tumor is bladder cancer, bone cancer, brain cancer, breast cancer, colorectal cancer, endometrial cancer, esophageal cancer, eye cancer, head and neck cancer, kidney cancer, lung cancer, melanoma, ovarian cancer, pancreatic cancer, prostate cancer, or gastric cancer, preferably breast cancer, lung cancer, renal cell carcinoma (RCC), hepatocellular carcinoma (HCC), melanoma, colorectal cancer, or endometrial cancer, particularly preferably primary or metastatic non-small cell lung cancer (NSCLC), and the metastatic cancer is metastatic melanoma, metastatic colorectal cancer, or metastatic endometrial cancer.

25. (canceled)

26. The method as claimed in claim 14, wherein the subject has recurrent cancer or refractory cancer; or wherein the use of hydroxyprogesterone acetate or the use of hydroxyprogesterone acetate in combination with an additional therapeutic agent for treating cancer reduces tumor cell proliferation in the subject and/or slows down or stabilizes tumor growth in the subject.

27. (canceled)
